(19) United States
(12) Patent Application Publication
(10) Pub. No.: US 2025/0177287 A1
(43) Pub. Date: Jun. 5, 2025

(54) METHODS AND SYSTEMS FOR INJECTABLE FORMULATIONS

(71) Applicant: Serán BioScience, LLC, Bend, OR (US)

(72) Inventors: Erica Schlesinger, Sisters, OR (US); Mark Kastantin, Bend, OR (US); Cindy Chung, Bend, OR (US); Mary Collins, Bend, OR (US); Dan Smithey, Bend, OR (US); Tanner Corrado, Bend, OR (US)

(21) Appl. No.: 18/963,333

(22) Filed: Nov. 27, 2024

Related U.S. Application Data
(60) Provisional application No. 63/605,430, filed on Dec. 1, 2023.

(51) Int. Cl. A61K 9/00; A61K 9/19; A61K 47/14

(52) U.S. Cl. CPC A61K 9/0019; A61K 9/19; A61K 47/14

(57) ABSTRACT

Systems and methods are provided for an injectable formulation. The injectable formulation includes a vehicle and dried particles. The dried particles have a tapped density greater than 0.45 mg/mL and are suspend in the vehicle at greater than or equal to 40 wt. %. The injectable formulation is injectable using a glide force of less than or equal to 100N.

### CROSS REFERENCE TO RELATED APPLICATIONS

The present application claims priority to U.S. Provisional Application No. 63/605,430 entitled “METHODS AND SYSTEMS FOR INJECTABLE FORMULATIONS”, filed Dec. 1, 2023. The entire contents of the above identified application is hereby incorporated by reference for all purposes.

### FIELD

The present description relates generally to physical properties of spray dried powders included in injectable formulations and methods of production for such powders.

### BACKGROUND/SUMMARY

It is not uncommon for some pharmaceutical formulations including certain types of active pharmaceutical ingredients (APIs), such as proteins, to demand delivery by injection. For such pharmaceutical formulations, administration by subcutaneous or intramuscular injection may be preferred. Subcutaneous or intramuscular injections may be performed at home, in some examples by using an auto-injector. At home administration improves patient experience and compliance and may also reduce health care costs. A formulation of an active pharmaceutical ingredient, such as a protein, delivered by subcutaneous or intramuscular injection often demands either a high concentration of API and/or a high injected volume to achieve target doses. Additionally or alternatively, an ocular injection may similarly demand the high concentration of API. One approach to increasing a dose concentration in an injectable pharmaceutical formulation is preparation of a suspension of microparticles including the API in a vehicle that does not solubilize the microparticles. The aforementioned approach has been demonstrated to achieve suspensions with API concentrations of up to 500 mg/mL or more. However, these suspension are highly viscous and the injection forces demanded for subcutaneous administration through an appropriately sized needle are greater than the injection forces acceptable for manual administration or forces supported by available auto-injectors.

In one example, the issues described above may be at least partially addressed by an injectable formulation including a vehicle and spray dried powder comprised of microparticles having a tapped density greater than 0.45 g/mL suspended in the vehicle at greater than or equal to 40 wt. % and wherein the injectable formulation is injectable through a 27 gauge, ½ inch length needle in a 1 mL pre-filled syringe at a rate of 1 mL in 10 seconds using a glide force of less than or equal to 100N. By targeting a threshold tapped density of the particles including the API, a bulk property of particles is identified for achieving the desired suspension viscosity and dispensing force. The tapped density of the spray dried particle may be impacted by particle morphology, particle size and distribution, and composition. As one example, the particles may be dried from solution by spray drying. A tapped density of dried particles may be increased by maximizing a concentration of the API in the solution. However, API stability and/or solution viscosity may limit a maximum API concentration. A tapped density of dried particles may be further increased by spray drying under vacuum. Spray drying under vacuum enables lower outlet temperatures and slower rates of drying that may result in an increase in tapped density that may be leveraged as an orthogonal method to increasing tapped density. Further spray drying under vacuum, or not under vacuum, while also adjusting atomization conditions may adjust an average particle size.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows a block diagram of a workflow for spray drying compounds at reduced pressures.

FIG. 2 shows a graph of glide force of a suspension of particles as function of particle tapped density.

FIG. 3 shows a chart comparing glide force of suspensions of particles having different average sizes.

FIG. 4 shows a chart comparing glide force of suspensions of particles including different concentrations of API.

FIG. 5 shows a graph of glide force of a suspension of particles as function of particle tapped density and SEM images corresponding to data points of the graph.

FIG. 6 shows a graph of glide force of a suspension of particles as a function of particle damped density including comparable data points of particles dried by standard spray drying and by vacuum spray drying.

FIG. 7 shows graphs depicting a correlation of Tₒᵤₜ and solids wt. % with tapped density of spray dried particles.

FIG. 8 shows a flow chart of a method for spray drying to obtain particles having at least a desired tapped density.

FIG. 9 shows graphs of glide force and viscosity of suspension of dried particles as a function of solids weight percent in the suspensions.

FIG. 10 shows graphs of glide force of suspensions of particles as function of particle tapped density for particles spray dried at atmospheric pressures.

FIG. 11 shows graphs of glide force of suspensions of particles as function of particle tapped density for particles spray dried at a reduced dryer pressure.

FIG. 12 shows a graph of glide force of suspensions of particles as function of outlet temperature.

FIG. 13 shows graphs of glide force of suspensions of particles as a function of solids weight percentage in the spray dried liquid feed.

FIG. 14 shows a graph of glide force of suspensions of particles as a function of particle tapped density for suspensions prepared at two different wt. % of particles.

FIG. 15 shows a graph of tapped density as function of droplet drying rate across a range of spray drying parameters.

### DETAILED DESCRIPTION

The following description relates to systems and methods for pharmaceutical formulations administered by injection. The pharmaceutical formulations may be a suspension of particles including an API, such as a peptide or protein, that demands administration by injection. Other examples of APIs include but are not limited to: antibodies or antibody fragments, peptides, enzymes, DNA/RNA or DNA/RNA fragments, protein degraders and small molecules. Small molecules may include chemically synthesized pharmaceutical ingredient typically <1500 Da. In order to prepare a suspension for which a glide force used to administer the injection from a needle and syringe is equal to or below a threshold glide force, the particles included in the pharmaceutical formulation may be above a threshold tapped density. The impact of tapped density on the glide force is dependent on the vehicle and solids concentration in suspension; tapped density has a significantly greater effect for suspensions that are approaching the maximum volume fraction in the suspension as defined by the Krieger & Dougherty equation (equation 1).

$$η = {η}_{0} {( 1 - \frac{φ}{{φ}_{max}} )}^{- [ η ] * {φ}_{max}} \qquad ( 1 )$$

η is viscosity of the suspension, η₀ is viscosity of the continuous phase (e.g., the vehicle), φ is volume of the dispersed phase (e.g., the solids) and φₘₐₓ is the maximum volume when viscosity diverges to infinity.

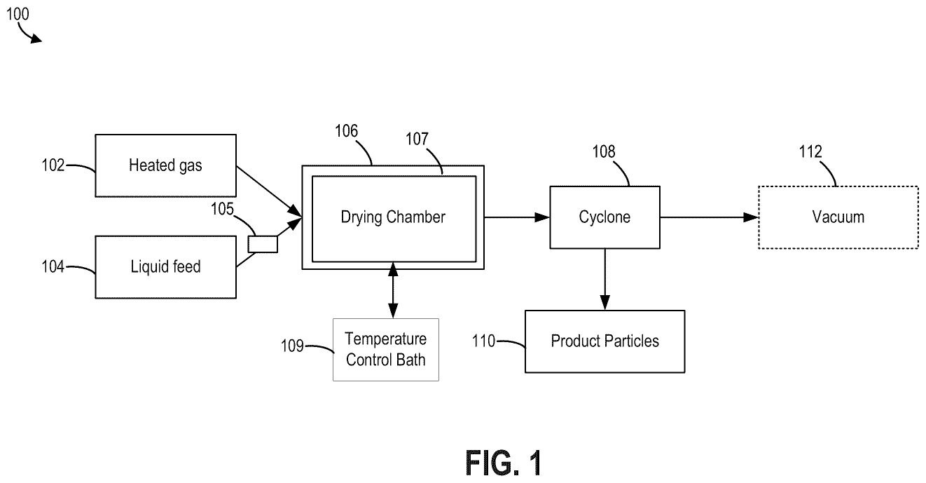

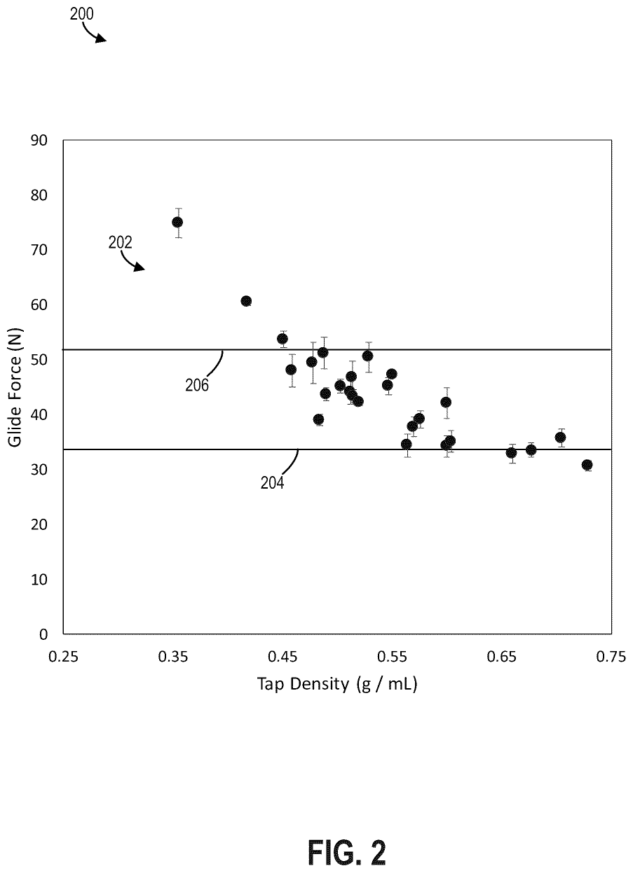

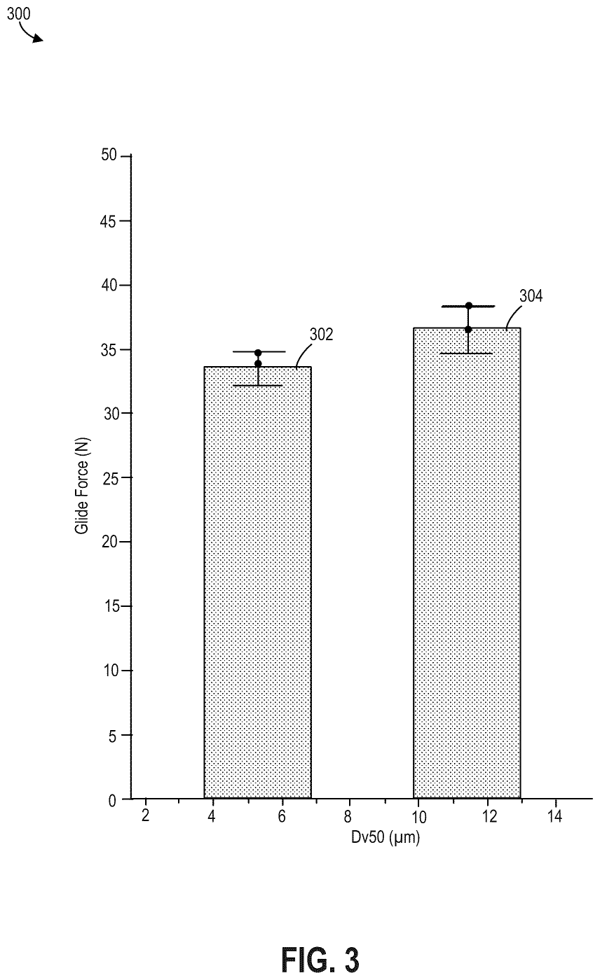

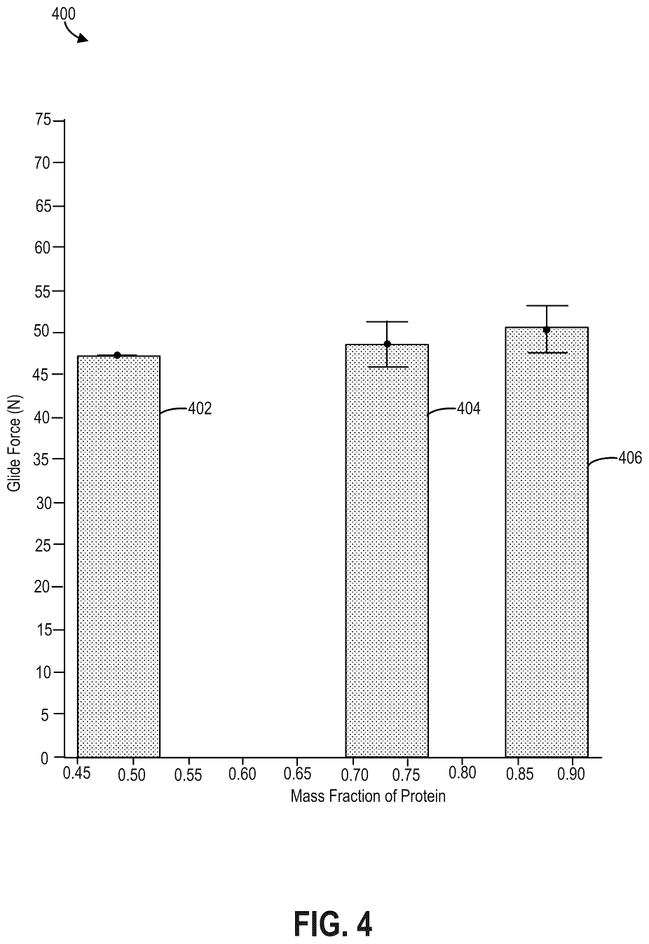

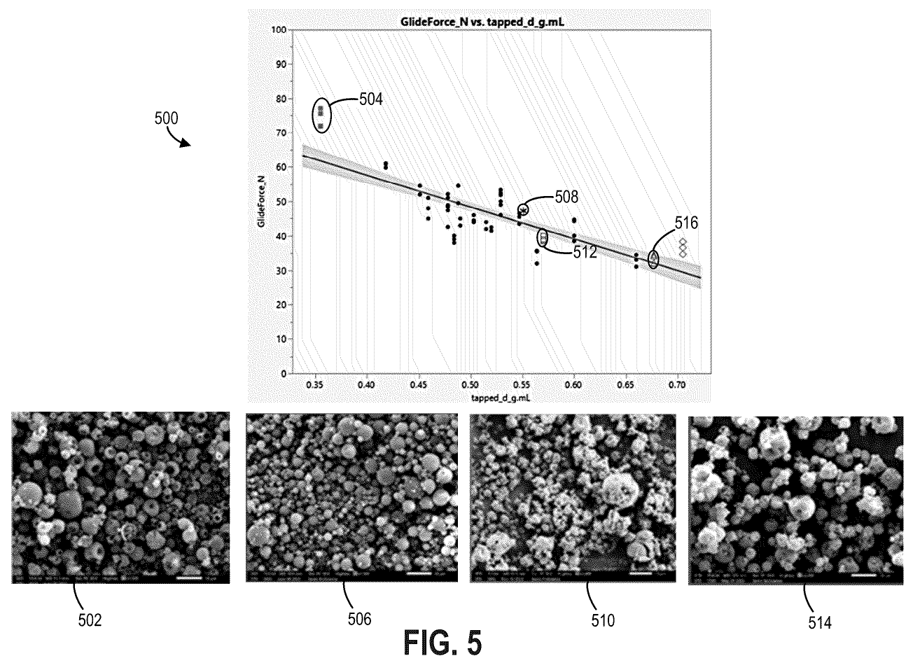

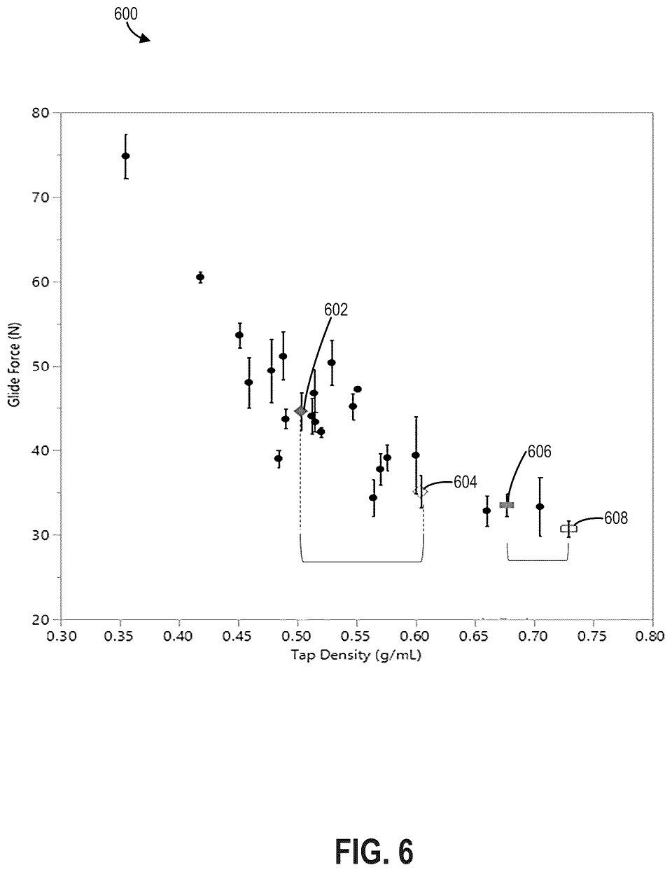

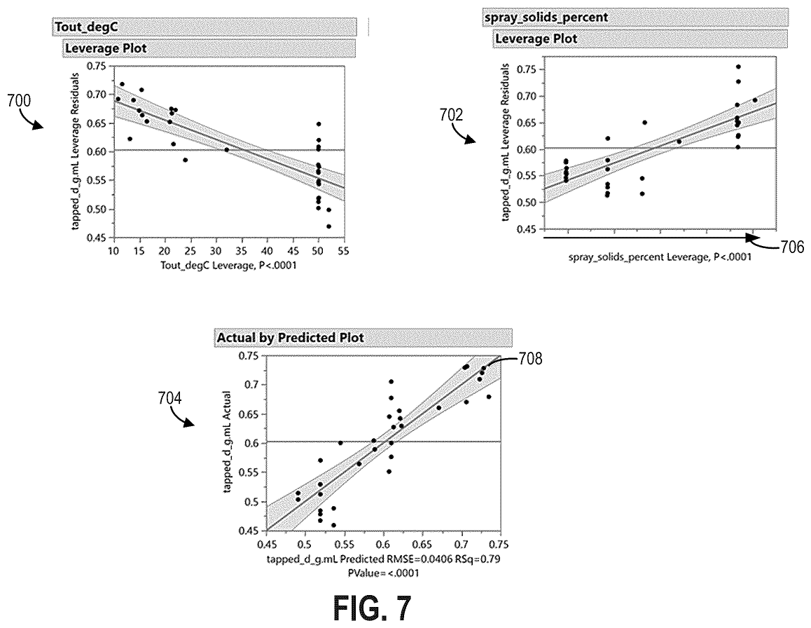

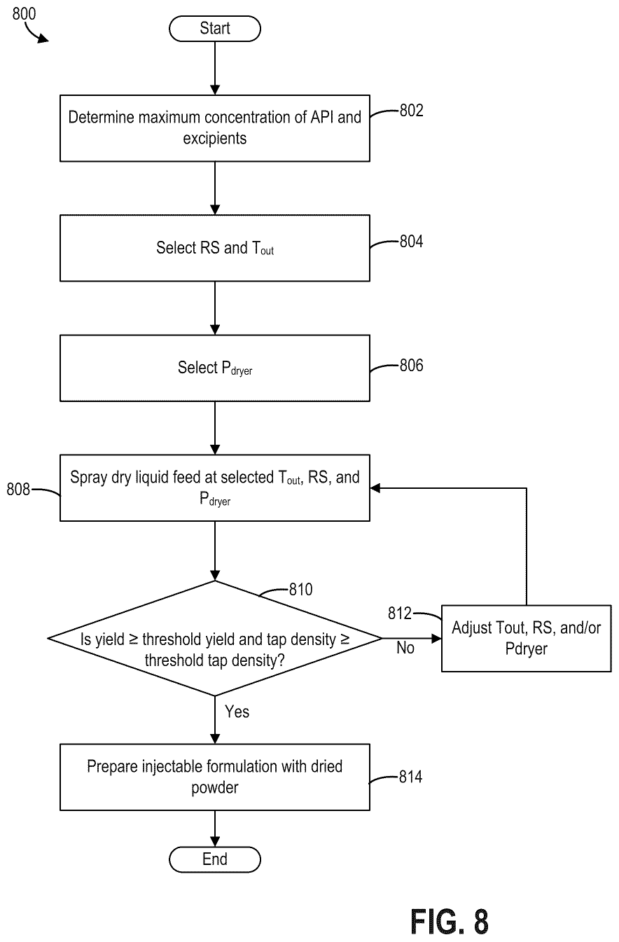

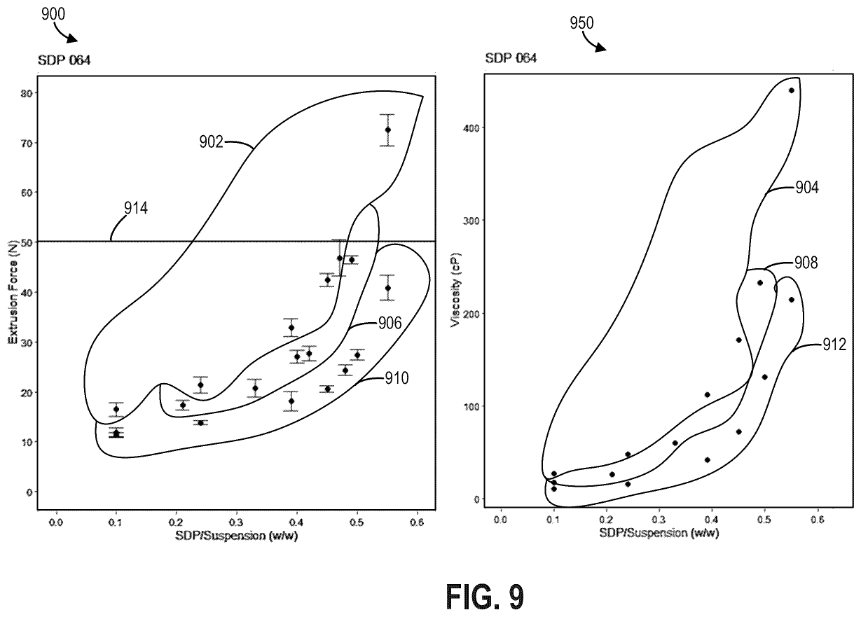

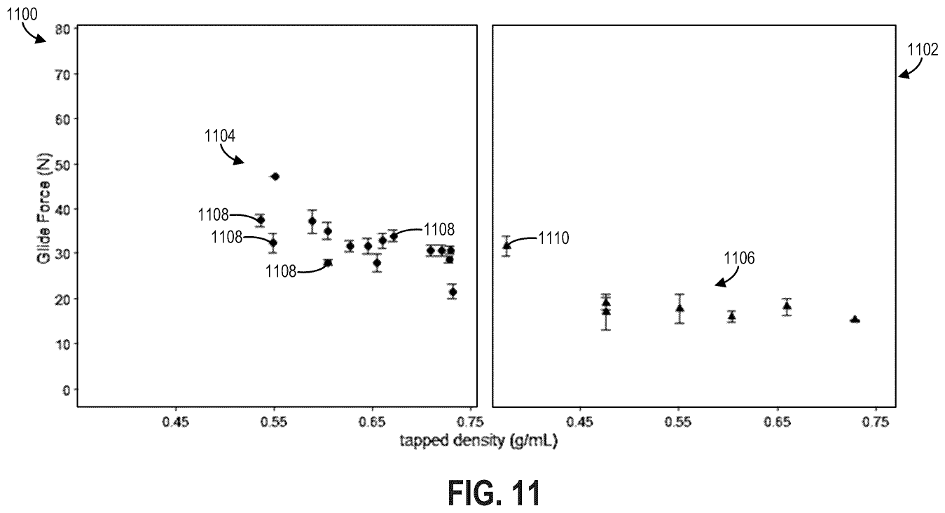

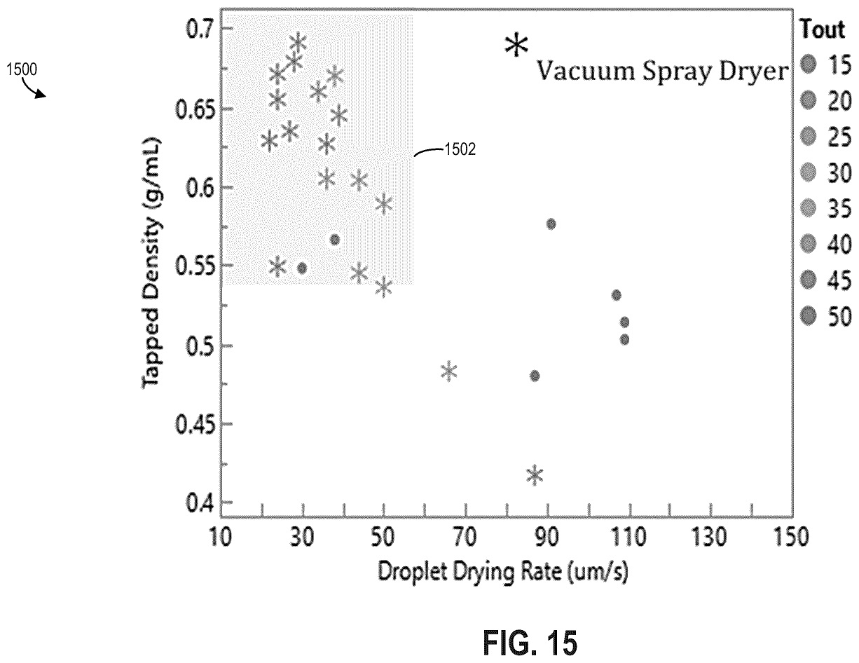

In some examples the threshold tapped density may be 0.45 g/mL. In some examples, the threshold glide force may be 50N. In alternate examples, the threshold glide force may be 100N. In some examples, powders at or above the threshold tapped density may be produced by drying a solution or suspension including the API via vacuum spray drying. A block diagram of an exemplary spray drying system is shown in FIG. 1. Powders comprised of dry particles including an API may have a characteristic tapped density. Spray drying parameters may be selected and adjusted to increase both tapped density and yield as shown in a method depicted as flow chart in FIG. 8. The spray drying parameters may be adjusted to maximize a tapped density of the dried particles as shown in the graphs of FIGS. 7 and 15. The multivariate analysis indicates that outlet temperature and spray solution concentration both have a statistically significant effect on tapped density of powders produced via spray drying. Glide force may be tested as a function of different spray drying parameters, suspension concentrations, and suspension vehicles as shown in FIGS. 9-14. When powders are incorporated into a high concentration suspension, the tapped density may correlate with glide force of the solution being pushed through a syringe, as shown in FIG. 2. Tapped density may be a more reliable indicator of resulting glide force of a suspension than other particle properties. Small variations in glide force of a suspension are shown when comparing powders of different d50 sizes, as shown in FIG. 3 or comparing powders including different concentrations of API as shown in FIG. 4. Additionally, comparing physical appearance of particles by SEM does not predict tapped density as shown in the graph of glide force and corresponding SEM images of FIG. 5. Vacuum spray drying, such as via the system shown in FIG. 1, enables spray drying at low outlet temperatures that may be beneficial to obtain dried particles having a higher tapped density. The graph shown in FIG. 6 compares tapped densities of particles obtained by conventional spray drying and by vacuum spray drying. Different types of APIs may be vacuum spray dried for inclusion in an injectable formulation. As an example, FIG. 11 illustrate that BSA, IgG and lysozyme proteins can all be spray dried under vacuum to yield both acceptable product yields and powder tap densities to achieve glide forces below 50N.

Turning now to FIG. 1, a schematic diagram of a spray drying system 100 is shown. In some examples, spray drying system 100 may include a vacuum 112 as described further below and may be a vacuum spray drying (VSD) system. Spray drying system 100 may include a drying chamber 106. Drying chamber 106 may be configured to receive a heated gas 102 and a liquid feed 104. In some examples, drying chamber 106 may be a jacketed drying chamber, including jacket layer 107 fluidly coupled to a temperature control bath 109. By setting a temperature of temperature control bath 109, a user may actively control a temperature of drying chamber 106. Under vacuum conditions, heated gas 102 may be flowed at a lower mass flow rate when compared to conventional (e.g., atmospheric pressure) spray drying. The lower mass flow rate may result in a larger influence of environmental heat exchange on an operating temperature (e.g., outlet temperature) of drying chamber 106. Jacket layer 107 and temperature control bath 109 may insulate drying chamber from heat exchange with the environment and provide increased accuracy in controlling the operating temperature of drying chamber 106.

Drying chamber 106 may be configured to receive a heated gas 102 and a liquid feed 104. Liquid feed 104 may be a suspension or solution including a liquid phase and a product either suspended or dissolved in the liquid phase. Spray drying system 100 may be configured to dry particles above a threshold tapped density. As one example, the product may be an active pharmaceutical ingredient (API) in a pharmaceutical formulation. The pharmaceutical formulation may include the API and other excipients. As one example, the API may be a pharmaceutically active protein (e.g., antibodies, enzymes, bacteriophages, cytokines, hormones, etc.). As another example, the API may include nucleic acids such as DNA or RNA. Types of RNA may include but are not limited to messenger RNA (mRNA), transfer RNA (tRNA), and/or short interfering (siRNA). As a further example, the API may be a self-assembled nanoparticle encapsulating a pharmaceutical compound. The self-assembled nanoparticle may be formed of lipids and/or block copolymers. In some examples, the self-assembled nanoparticle may not be an API, but may be a carrier for the API. For example, a lipid nanoparticle encapsulating an RNA fragment may be spray dried. As an additional example, the product may be an amorphous solid dispersion of an API. The aforementioned APIs as well as other APIs may be preferentially administered to a patient by subcutaneous or intramuscular injection. For subcutaneous or intramuscular administration, a suspension including the API at or above threshold dosage that results in a glide force through a needle and syringe/cartridge appropriate for subcutaneous, ocular, or intramuscular delivery at or below a threshold glide force may be desired. As one example, the API may be a protein and a threshold dosage may be 200 mg/mL. As an alternate example, the threshold dosage may 500 mg/mL. In some examples, the threshold dosage may be greater than 500 mg/mL. As a further example, a threshold glide force may be 50N. In alternate examples, the threshold glide force may be 100N. A suspension of the API having the aforementioned properties may be prepared from particles having a high tapped density produced by a vacuum spray drier. Additionally, spray drying using a vacuum spray dryer may enable spray drying a low Tₒᵤₜ, which in addition to promoting high tapped density, may also be preferred for the aforementioned APIs which may degrade at elevated temperatures.

The liquid phase of the spray solution may be water or a high volatility organic solvent having a boiling point of 100° C. or lower (e.g., acetone, methanol, ethanol, isopropyl alcohol, ethyl acetate, etc.), or some combinations thereof. As one example, the liquid phase may be 100% water. As an alternate example, the liquid phase may be between 50% and 100% water by weight and less than or equal to 50% organic solvent by weight. As a further example, the liquid phase may include between 5%-100% low-volatility organic solvent. The low volatility organic solvent may be an organic solvent having a boiling point above 150° C. at standard pressure (e.g., 1 atm) such as dimethyl sulfoxide (DMSO), dimethylformamide (DMF), N,N-dimethylacetamide (DMAc) or N-methyl-2-pyrrolidone (NMP). A remainder of the liquid phase may be comprised of a miscible co-solvent which may be water or a high volatility organic solvent.

Heated gas 102 may be air or nitrogen, for example. Nitrogen may be used as heated gas 102 when the product is prone to oxidation or if the liquid phase is flammable. Heated gas 102 and liquid feed 104 may be introduced into drying chamber 106. Liquid feed 104 may enter drying chamber 106 through an atomizer 105. Atomizer 105 may be configured to disperse liquid feed 104 into small droplets, thereby maximizing an amount of liquid surface area exposed to heated gas 102. Liquid feed 104 and heated gas 102 may enter drying chamber 106 at varied geometries. In one embodiment, heated gas 102 and liquid feed 104 may both enter at a top of drying chamber 106 in a co-current configuration. In an alternate embodiment, liquid feed 104 may enter the top of drying chamber 106 while heated gas 102 enters drying chamber 106 from a bottom of drying chamber 106 in a counter-current configuration.

Operating parameters of spray drying system 100 may be chosen such that interaction of heated gas 102 with the atomized droplets of liquid feed 104 results in evaporation of the liquid phase by the time the atomized droplets exit drying chamber 106. Operating parameters may include pressure within drying chamber 106, temperature at an outlet of drying chamber 106, and a ratio of liquid feed 104 to heated gas 102.

The product may exit drying chamber 106 and may enter cyclone 108. Cyclone 108 may be configured to separate product particles 110 from the heated gas through vortex separation. Product particles 110 may be collected from an outlet of cyclone 108. Vacuum 112 may be fluidly coupled to drying chamber 106 via cyclone 108. In this way, vacuum 112 may evacuate drying chamber 106 and thereby reduce an operating pressure within drying chamber 106. Sizing of cyclone 108, positioned between vacuum 112 and drying chamber 106, may be chosen based on a balance between a desired operating pressure of a VSD and a desired collection efficiency. In one example, a larger cyclone may reduce a pressure drop between an inlet of cyclone 108 and an outlet of cyclone 108 and a load on vacuum 112 may be decreased. A smaller cyclone may collect particles, especially smaller particles, more efficiently than a larger cyclone. For this reason, a smaller cyclone may be chosen if a smaller average particle size distribution is desired.

The collected product may be a dried powder including the API. The dried powder may be included in a non-aqueous suspension configured to be administered to a patient subcutaneously, ocularly, or intramuscularly. To reach a target API dosage and syringe glide force, the particles comprising the suspension are dried to have a tapped density above a threshold tapped density. The tapped density may capture both bulk particle properties and particle-particle interactions, both influencing suspension viscosity and thus dispensing force from a syringe.

A method 800, for spray drying particles having a high tapped density is shown as a flow chart in FIG. 8. The spray drying may be carried out on a spray drying system such as spray drying system 100 as shown in FIG. 1. The spray drying system may be used in a co-current or counter-current configuration.

At 802, method 800 includes determining a maximum concentration of API and other excipients in the liquid feed. The maximum concentration in the liquid feed may include an API and other excipients dissolved in an appropriate solvent or solvent blend. The solvent or solvent blend may be chosen for ease of spray drying and/or compatibility with the API and other excipients. As one example, the maximum concentration of the API in the liquid feed may be dictated by a solubility of the API in the solvent or solvent blend, stability of the API (e.g., protein stability) in the solvent or solvent blend, and/or viscosity of the resulting liquid feed solution. Selecting a maximum concentration of liquid feed may increase an amount of solid per spray droplet, thereby increasing tapped density of the resulting dried particles.

At 804, method 800 includes selecting a relative saturation (RS) and Tₒᵤₜ of the spray dryer. Selecting a lower temperature and higher relative saturation may further increase particle density. Decreasing a Tₒᵤₜ and increasing relative saturation may delay an onset of particle skinning, thereby increasing a particle density and contributing to an increase in tapped density. However, high relative saturation and low Tₒᵤₜ may also decrease a drying rate and decrease a yield of dried particles. Because high concentration of API favors high density particles, Tₒᵤₜ may be selected to be higher and RS may be selected to be lower for a liquid feed with a higher max API concentration than a liquid feed with a lower max API concentration in order to balance low void volume with a desired yield.

Turning briefly to FIG. 7, a first graph 700 shows measured tapped density of collected spray dried powders as a function of Tₒᵤₜ during spray drying. As shown in graph 700, decreasing Tₒᵤₜ is strongly correlated with increasing tapped density. A second graph 702 shows tapped density of collected spray dried powders as a function of spray solids wt. % in the liquid feed. An arrow 706 indicates a direction of increasing solids wt. % along an x-axis of graph 702. As shown in graph 702, increasing solids wt. % is strongly correlated with increasing tapped density. Based on the correlations of graph 700 and 702, a tapped density may be predicted for the product (e.g., API) spray dried to obtain the data points of graph 700 and 702 based on the solids wt. % and selected Tₒᵤₜ. From the data graphed in graphs 700 and 702, accuracy of the prediction is shown by graph 704 of measured tapped density as a function of predicted tapped density. A line 708 of graph 704 corresponds to a perfect 1:1 prediction of tapped density. The data correlates well with line 708, confirming the above discussed effects of Tₒᵤₜ and solids wt. % on tapped density of spray dried particles.

Turning briefly to FIG. 15, a plot 1500 of tapped density as function of calculated drying rate (e.g., droplet drying rate) may also show close to a linear correlation and may be used to predict tapped density. The calculated drying rate may encompass both Tₒᵤₜ and relative saturation into a parameter that can be used to adjust the tapped density of the resulting spray dried powder. Asterisk (*) shaped data points indicate data collected by vacuum spray drying and circular data points indicate data collected by conventional spray drying. Color gradients correspond to data collected at Tₒᵤₜ in range of 15° C. to 50° C. Data points within box 1502 correspond to data collected at Tₒᵤₜ in a range of 15° C. to 30° C. and data points outside of box 1502 correspond to data collected at Tₒᵤₜ in a range of 35° C. to 50° C. As shown in plot 1500, tapped density and droplet drying rate show a close to linear relationship. In this way a calculated droplet dry rate may be proportional to a tapped density of the resulting spray dried powder. Further, a calculated droplet rate may be used to anticipate and/or predict a tapped density of the resulting spray dried powder.

Returning now to FIG. 8, Tₒᵤₜ may be less than room temperature. In some examples Tₒᵤₜ may be less than a boiling point of the solvent or a majority (e.g., >50% by volume) of the solvent blend. In some examples, Tₒᵤₜ may be less than 40° C. In further examples Tₒᵤₜ may be less than 30° C. RS may be higher than a conventional target RS for spray drying from aqueous liquid feeds. As one example RS may be in a range of 5%-20%. As an alternate example, RS may be in a range of 5%-50%. In a further example, RS may be greater than 10%. In some examples, RS and Tₒᵤₜ may be selected together. For examples Tₒᵤₜ may be less than 40° C. and RS may be >10%.

At 806, method 800 includes selecting P_{dryer}. In examples where the Tₒᵤₜ is low enough and/or RS is high enough to decrease spray drying yield below an acceptable level, P_{dryer} may be decreased to compensate. As one example P_{dryer} may be less than an atmospheric pressure of the spray dryer. As a further example, P_{dryer} may be less than 0.6 bar or less than 0.8 bar. In some examples P_{dryer} may be less than 0.6 bar or less than 0.8 bar when Tₒᵤₜ is less than 35° C. As a further example, P_{dryer} may be less than 0.6 bar or less than 0.8 bar when Tₒᵤₜ is less than 50° C. and RS is greater than 5%.

At 808, method 800 includes spray drying the liquid feed at the selected Tₒᵤₜ, P_{dryer}, and RS. Method 800 then proceeds to 810 and includes determining if the spray dried powders are at or above a target tapped density or a suspension of the spray dried powder is dispensed at or below a threshold glide force, and at or above a threshold yield. Target tapped density may be measured using conventional lab equipment (e.g., a graduated cylinder and a balance) as described in United States Pharmacopia (USP) <616>. As one example, a threshold tapped density may be 0.45 mg/mL. As a further example, the tapped density may be in a range of 0.5 g/mL to 0.8 g/mL. In alternate examples, tapped density may be in a range of 0.65 to 0.75 g/mL. A threshold yield may depend on a scale (e.g., total volume of liquid feed) of spray drying, among other things. As one example a threshold yield may be 45%. In some examples, 808 may additionally include determining if a particle size distribution of the spray dried particles is within a threshold particle size distribution range. Determining a desired particle size (e.g., D_{V,90}) threshold range is discussed further below with respect to FIG. 3.

If at 810 it is decided that a desired tapped density and target yield are not at or above desired values, method 800 proceeds to 812 and adjusting Tₒᵤₜ, RS, P_{dryer}, and/or maximum spray solution concentration of the liquid feed. For example, if the dried particles are above the threshold tapped density but below a threshold yield, P_{dryer} may be decreased while keeping Tₒᵤₜ and RS constant. As an alternate example, if the dried particles are above a threshold yield but below a threshold tapped density, Tₒᵤₜ may be decreased and RS may be increased while keeping P_{dryer} constant. In further examples, adjusting may include decreasing Tₒᵤₜ, increasing RS while also decreasing P_{dryer}. Method 800 then returns to step 808 and spray drying of the liquid feed is performed at the adjusted Tₒᵤₜ, RS, and P_{dryer}.

If at 810, it is decided that dried powder (e.g., spray dried particles) are at or above the threshold yield and threshold particle density, method 800 proceeds to 814 and includes preparing an injectable formulation with the dried particles. The prepared injectable formulation may be deliverable by an auto-injector, a pre-filled syringe, or a standard needle and syringe. Preparing an injectable formulation may include suspending the dried particles in a suitable solvent (e.g., vehicle). In examples where the API is soluble in water (e.g., proteins) a non-aqueous vehicle may be preferred. The non-aqueous vehicle may be selected from Federal Drug Administration (FDA) approved vehicles based on a viscosity, density, and compatibility of the vehicle with the API, proteins, and injection device materials. As one example, the vehicle may be a mix of glyceryl tricaprylate/tricaprate, triacetin, ethyl oleate, or propylene glycol dicaprylate/dicaprate, combinations of the aforementioned vehicles, or other proprietary compositions, among others.

An API concentration in the injectable may be high enough that a desired dosage of the API may be delivered in a single fast injection. For example, the desired dosage may be delivered in a range of 1 mL to 2 mL of suspension. Further, the API concentration in the injectable formulation may be greater than 200 mg/mL or may be greater than 500 mg/mL. The injectable formulation may include the spray dried powder at greater than or equal to 40 wt. %. In some examples, the injectable formulation may include the spray dried powder at greater than or equal to 50 wt. %. In further examples, the injectable formulation may include the spray dried powder at greater than or equal to 60 wt. %. A threshold dispensing force (e.g., glide force) for dispensing the injectable formulation may be less than or equal to 50N or less than or equal to 100N at a speed of greater than or equal to 0.1 mL/second (e.g., 1 mL over 10 seconds) or greater than or equal to 1 mL over 8 seconds through a 27 gauge ½ inch long needle. In some examples, a wt. % of the spray dried powder may include as the threshold dispensing force increase. For example, an injectable formulation with a threshold glide force less than or equal to 50N may include spray dried powder at greater than or equal to 40 wt. %. Alternatively, an injectable formulation with threshold glide force less than or equal to 100N may include spray dried powder at greater than or equal to 50 wt. % or greater than or equal to 60 wt. %. Additionally, the injectable formulation may be stable at less than or equal to 25° C. for up to two years. In some examples, the injectable formulation may be stable for up to 2 years at temperatures in a range of 2° C. to 8° C. Method 800 ends.

Turning now to FIG. 9, it shows a first graph 900 shows glide force as a function of wt. % of spray dried particles in suspension and second graph 950 shows a viscosity as a function wt. % of spray dried particles in the suspensions. The suspensions measured with respect to glide force in first graph 900 are the same suspensions measured with respect to viscosity in second graph 950. The spray dried particles include BSA as a model protein and also include an equivalent weight of sucrose. For glide force measurements the suspension is pushed through a 27 gauge ½ inch length special thin-wall (STW) needle at a rate of 0.125 mL/sec.

First graph 900 includes a data set 902 and second graph 950 includes a data set 904. Data sets 902 and 904 (red markers) each correspond to a suspension prepared using a glyceryl tricaprylate/tricaprate vehicle. First graph 900 includes a data set 906 and second graph 950 includes a data set 908 (blue markers). Data sets 906 and 908 each correspond to a suspension prepared using glycerol triacetate as the vehicle. First graph 900 includes a data set 910 and second graph 950 includes a data set 912 (green markers). Data sets 910 and 912 each correspond to a suspension prepared using propylene glycol dicaprylate/dicaprate.

Comparing graph 900 to graph 950, data sets 902, 906, and 910 each follow a similar trend with respect to increasing suspension wt. % as data sets 904, 908, and 912. For this reason, suspension viscosity may dictate an extrusion force. As one example, 250 cP, measured at a shear rate of 2000 s⁻¹ may be a maximum viscosity corresponding to a threshold glide force of 50 N (indicated by line 914 on graph 900). Further, graph 950 shows that a magnitude of increase in viscosity with respect to increasing solids loading may be different for different vehicles. In alternate examples, the threshold glide force may be 100N. In such examples, a viscosity of the suspension may be less than or equal to 500 cP measured at a rate of 2000 s⁻¹.

Turning now to FIG. 2, it shows a graph of glide force as a function of tapped density. A plurality of data points 202 correspond to a tapped density of a dried powder and a glide force used to push a 40 wt. % suspension of spray dried powder including BSA and sucrose through a 27 gauge ½ inch long STW needle from a 1 mL pre-filled syringe at an injection speed of 0.125 mL/min. The plurality of data points 202 show a negative correlation between glide force and tapped density up to a minimum glide force indicated by line 204. As shown by graph 200, glide force may be decreased by about 60% by increasing a tapped density of the dried particles included in the injectable formulation. A second line 206 corresponds to an example of an upper threshold glide force for subcutaneous or intramuscular administration. The suspensions with higher tapped density may include fewer particles than the suspensions with lower tapped density particles. The decrease in total number of particles may decrease a viscosity of the suspension and therefore decrease the glide force demanded for injection. By increasing the tapped density, glide force of suspensions incorporating the dried particles may be tuned to be below the threshold glide force. In this way, dried particle properties may be tuned and an injectable suspension may be produced without tuning other suspension properties, such as particle concentration or by suspension additives, such as viscosity reducers, which may increase an overall cost of the suspension or destabilize the API. Additionally, viscosity reducers may reduce stability (e.g., increase a degradation rate) of the suspension and reducing or eliminating a demand for viscosity reducers may therefore increase stability of the suspension.

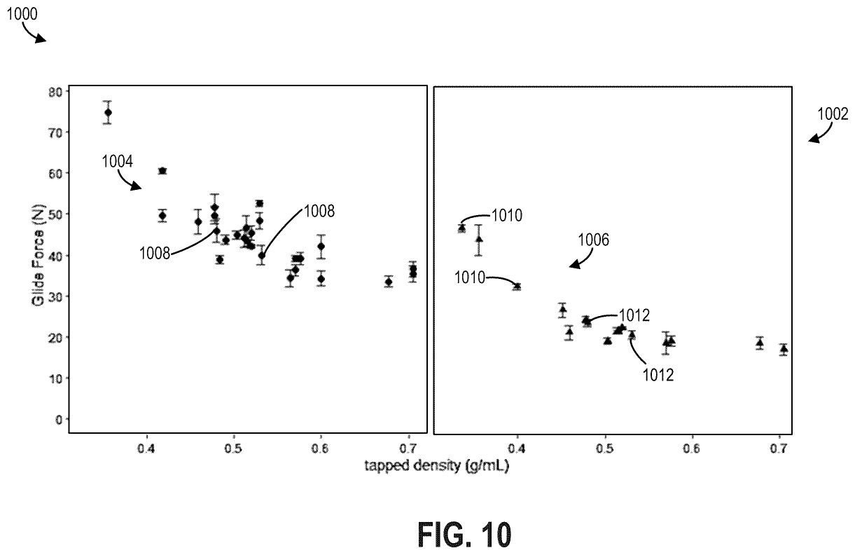

Turning now to FIG. 10, it shows a first graph 1000 of glide force demanded for injection of a suspension of 40 wt. % particles through a 27 gauge ½ inch long STW needle at an injection speed of 0.125 mL/second for particles spray dried at atmospheric pressure and Tₒᵤₜ>40° C. as a function of tapped density of the particles. Data set 1004 corresponds to spray dried particles including BSA suspended in a glyceryl tricaprylate/tricaprate vehicle. Graph 1000 also shows data for lysozyme suspended in glyceryl tricaprylate/tricaprate shown by markers 1008. The trend of decreasing glide force with increasing tapped density is not dependent on a type of active ingredient (or model active ingredient) included in the particle.

Second graph 1002 of FIG. 10 shows glide force as function of tapped density for spray dried particles dried in substantially the same way as particles for first graph 1000 and prepared as a suspension and dispense in substantially the same way as in first graph 1000. Second graph 1002 includes data set 1006 corresponding to spray dried particles including BSA. Spray dried particles including IgG 1010, and spray dried particles including lysozyme 1012 are also shown. Each suspended in a propylene glycol dicaprylate/dicaprate. Comparing data set 1004 and data set 1006, the relationship between glide force and tapped density is similar, regardless of the vehicle used. However, the magnitude of decrease in glide force as tapped density increases may depend on the vehicle and the suspension concentration. For example, a larger change in glide force as function of tapped density is observed in the data shown in graph 1000 where the vehicle is glyceryl tricaprylate/tricaprate than in data shown in graph 1002 where the vehicle is propylene glycol dicaprylate/dicaprate.

Turning now to FIG. 11, it shows a first graph 1100 and a second graph 1102. Both first graph 1100 and second graph 1102 show glide force as a function of tapped density for particles prepared by spray drying at a pressure below atmospheric pressure and at Tₒᵤₜ<40° C. Glide force is measured from a 40 wt. % suspension of particles through a 27 gauge ½ inch long STW needle at an injection speed of 0.125 mL/second. First graph 1100 includes a data set 1104 corresponding to spray dried particles including BSA and data points 1108 corresponding to spray dried particles including lysozyme, each suspended in a glyceryl tricaprylate/tricaprate vehicle. Second graph 1102 includes a data set 1106 corresponding to spray dried particles including BSA, and data points 1110 corresponding to spray dried particles including IgG, each suspended in a propylene glycol dicaprylate/dicaprate.

Comparing data set 1104 to data set 1106, a similar comparison can be observed with respect to a change in magnitude of the dependence of glide force on tapped density when the vehicle of the suspension is changed. Further, comparing data set 1004 of FIG. 10 to data set 1104 shows a general shift of particle tapped density to higher tapped density when P_{dryer} and Tₒᵤₜ are both decreased. A similar comparison can also be made between the data of graph 1100 of FIG. 10 and the data of graph 1102. Further, a maximum tapped density achieved for spray dried particles may be increased by decreasing P_{dryer} and Tₒᵤₜ. For example, a maximum tapped density for data sets shown in FIG. 10 is 0.70 g/mL while a maximum tapped density for data sets shown in FIG. 11 is 0.73 g/mL.

As described above with respect to method 800, a concentration of API in the liquid feed may be maximized to increase particle and powder density. However, a maximum concentration of the API may be limited due to solubility, stability, or solution viscosity and may demand additional tuning of spray drying parameters such as Tₒᵤₜ, RS, and P_{dryer}. to optimize particle and powder tapped density.

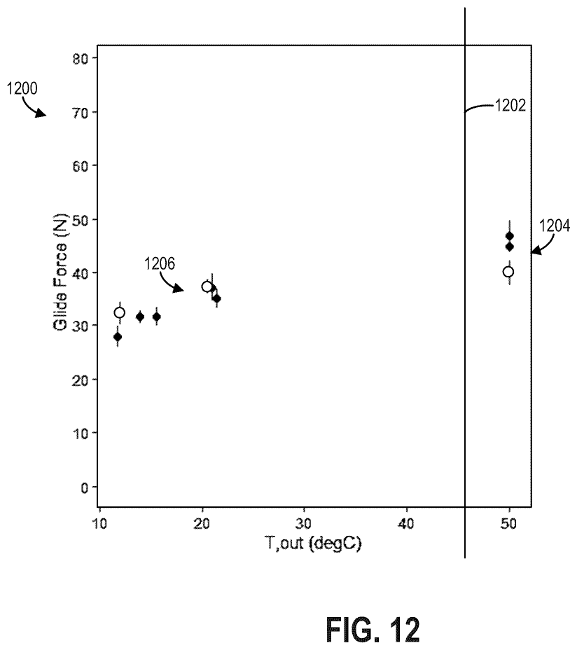

Turning now to FIG. 12, it shows a graph 1200 of glide force as function of Tₒᵤₜ measured for injection of a suspension including the spray dried particles at 40 wt. % in glyceryl tricaprylate/tricaprate dispensed as described above with respect to FIGS. 10 and 11. The spray dried particles may include BSA (filled circles) or lysozyme (open circles) as a model API. Spray drying liquid feeds of both BSA and lysozyme are prepared with liquid feeds with <9 wt % of solid material, including the model API.

Line 1202 delineates Tₒᵤₜ that is selected for spray drying at atmospheric pressure (>40° C.°) from Tₒᵤₜ that is selected for spray drying a P_{dryer} less than atmospheric pressure (<40° C.). First data set 1204 to the right of line 1202 corresponds to suspension of particles obtained by spray drying at 50° C. at atmospheric pressure. If it is desired to decrease glide force from what is measured for first data set 1204 but increasing solids concentration is not possible, as described above with respect to method 800, decreasing Tₒᵤₜ may slow a drying rate, resulting in denser particles. Decreasing Tₒᵤₜ may subsequently demand a decrease in P_{dryer} to maintain a desired yield. Second data set 1206, to the left of line 1202, corresponds to suspensions of particles obtained by spray drying at Tₒᵤₜ<40° C. and P_{dryer} less than atmospheric pressure. By decreasing the Tₒᵤₜ the glide force for the suspension of the resulting particles is decreased. The decreased glide force may be due to the increase in density achieved by slowing the spray drying rate by decreasing Tₒᵤₜ.

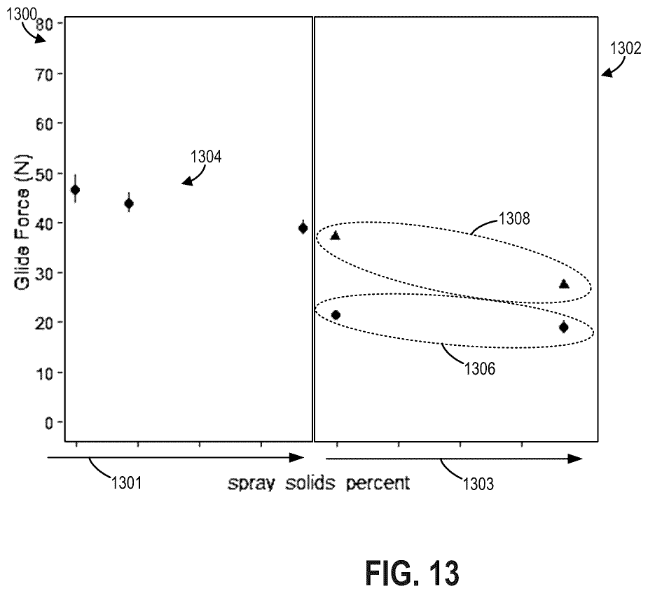

Turning now to FIG. 13, the effect of solids loading in a spray solution on glide force is shown. FIG. 13 includes a first graph 1300 and a second graph 1302, both of which show glide force of a suspension including spray dried particles as function of solids weight percent in the liquid feed used to prepare the spray dried particles. An arrow 1301 indicates a direction of increasing solids weight percent along the x-axis of first graph 1300 and an arrow 1303 indicates a direction of increasing solids weight percent along the x-axis of second graph 1302. Glide force of first graph 1300 and glide force for second graph 1302 were each measured by injecting through a 27 gauge ½ inch long STW needle at an injection speed of 0.125 mL/sec.

Data set 1304 of graph 1300 corresponds to particles suspended in glyceryl tricaprylate/tricaprate. Data set 1304 shows that as wt. % of spray solids increases the corresponding glide force decreases. The decreased glide force may be caused by a corresponding increase in tapped density as the amount of solids in the spray solution increases as described above with respect to FIG. 7.

Second graph 1302 includes a second data set 1306 and a third data set 1308. Second and third data sets 1306 and 1308 may each correspond to suspensions of particles suspended in propylene glycol dicaprylate/dicaprate. Second data 1306 set further corresponds to glide force of a 40 wt. % suspension of spray dried particles and third data set 1308 corresponds to glide force of a 50 wt. % suspension of spray dried particles. Regardless of suspension wt. %, the same trend of glide force as a function of solids wt. % is seen as in first graph 1300. Additionally, comparing second data set 1306 to third data set 1308, a magnitude of decrease in glide force as function of solids loading percent is greater as the weight of particles in the injected suspension is increased.

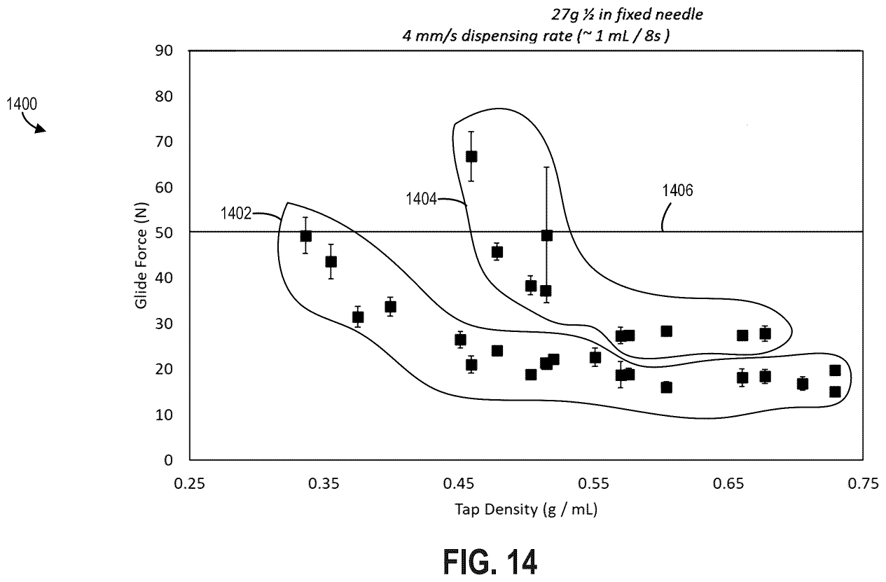

Turning now to FIG. 14, it shows a graph 1400 of glide force as function of tapped density for two suspensions. A first suspension is a 40 wt. % suspension of spray dried particles and a second suspension is 50 wt. % suspension of spray dried particles. Both the first suspension and the second suspension were prepared using a propylene glycol diester vehicle and dispensed through a 27 gauge ½ inch long STW needle at a rate of approximately 0.1 mL/s. A line 1406 denotes a 50N threshold.

A first data set 1402 corresponds to measured glide forces of the first suspension and a second data set 1404 corresponds to the second suspension. As shown in graph 1400, by preparing the second suspension with particles having a high (e.g., >0.45 g/mL) tapped density a weight percent of the particles in the suspension may be increased without the glide force being above the 50N threshold.

Tapped density of a dried powder may correlate to glide force of the syringed suspension including the dried powder more closely than other physical properties of the dried powder. For example, as shown in graph 300 of FIG. 3, for a subset of the suspension of graph 200, glide force is compared based on particle size instead of tapped density. A first column 302 corresponds to dried particles having a D_{v,50} (e.g., average particle size) between about 4 μm and about 7 μm. A second column 304 corresponds to particles having a D_{v,50} between about 10 μm and about 13 μm. A height of first column 302 and second column 304 corresponds to a glide force indicated along the y-axis of graph 300. Despite column 304 corresponding to particles about twice as large as the particles as the particles corresponding to column 302, glide force increases by a relatively small amount.

A glide force of the suspension may not be closely dependent on particle size, instead, particle size may be selected based on the diameter of needle delivering the suspension. For example, an upper D_{v,90} four times smaller than an inner diameter of the needle may be desired. As one example, an inner diameter of a 27 g needle may be 0.2 mm and an upper D_{v,90} may be less than or equal to 50 μm. As a further example, an upper limit of particle size may be a D_{v,90} of 100 μm. A lower limit of particle size may dependent on a collection yield of the drying system. As a one example, a lower limit of particle size may be a D_{v,10} of 1 μm.

Additionally, tapped density of the dried powder may influence glide force more strongly than an overall concentration of the API in the dried powder. For example, as shown FIG. 4 a graph 400 compares glide force for suspensions of the same dried particles at different protein concentrations. A first column 402 corresponds to a formulation including about 0.45 to 0.53 mass fraction of a protein with respect total solids in the liquid feed (e.g., protein plus other excipients). A second column 404 corresponds to a formulation including about 0.68 to about 0.77 of a protein. A third column 406 corresponds to a formulation including about 0.84 to about 0.92 of a protein. A height of the columns along the y-axis of graph 400 corresponds to a glide force of an injection including the formulation. The height of first column 402, second column 404 and third column 406 do not change by more than 10% despite that the percent of protein corresponding to column 406 is nearly double that of first column 402.

Additionally, tapped density may be easier to quantify and more closely related to glide force than observed physical characteristics of the dried particles, such as may be seen in a scanning electron microscopy (SEM) image. For example, FIG. 5 shows a graph 500, similar to graph 200 of FIG. 2 plotting data points corresponding to glide force as a function of tapped density of the dried particles included in the suspension. A first SEM image 502 corresponds to a first set of data points 504. A second SEM image 506 corresponds to second set of data points 508. A third SEM image 510 corresponds to a third set of data points 512. A fourth SEM image 514 corresponds to a fourth set of data points 516. The surface morphology of the particles shown in the first, third, and fourth SEM images are very similar despite the first, third, and fourth SEM images each corresponding to significantly different glide forces, as shown on graph 500. Second SEM image 506 shows a collection of particles having a relatively smooth morphology compared to the particles of the first, third, and fourth SEM images. The particles of image 506 may be described as substantially free of wrinkles, dimples, and holes. However, data points 508 corresponding to second SEM image 506 have a higher glide force than data points corresponding to the third and fourth SEM images which show a rougher, more wrinkled morphology and a lower glide force than data points corresponding to the first image which also shows wrinkled morphologies. In this way tap glide force of the resulting suspension is shown to not be strongly correlated with surface morphology of the particles.

Additionally, image analysis may be used to quantify a percent composition of particles comprising powder sample with respect to a volume percent of hollow particles, a volume percent of internal voids, and volume percent of solid particles (e.g., solid particles without internal voids). For example, image analysis may be performed using X-ray computed tomography to create a 3-D representation of a powder sample. Hollow particles may refer to bubble shaped particles having a solid exterior with a central void. Internal voids may refer to the volume of air enclosed in the bubbles. Table 1 below compares a volume percent of each phase listed above for two different spray dried powder samples in addition to the measured tapped density.

TABLE 1
Particle properties and tapped density
of spray dried particle samples

| | | |
|---|---|---|
| Phase | Sample 1 | Sample 2 |

Volume (%)

| | | |
|---|---|---|
| Internal Voids | 3.0 | 1.0 |
| Solid Particles | 52.3 | 90.9 |
| Hollow Particles | 44.7 | 8.1 |

Powder Properties

| | | |
|---|---|---|
| Tapped Density (g/mL) | 0.729 | 0.503 |

Table 1 illustrates that geometric arguments alone may be insufficient to explain differences in tapped density. Sample 1 was shown to include more internal voids and hollow particles than sample 2 as percentage of the total particle volume and thus this analysis may predict that tapped density for Sample 1 may be higher than Sample 2. However, the tapped density of Sample 1, when measured, is significantly higher than Sample 2. Furthermore, as shown in FIG. 2, tapped density of a powder correlates well with the ability to create a high concentration suspension from the powder. This illustrates the ability of tapped density to incorporate additional properties of the powder into a single measureable parameter that is predicative of the injectable suspension. Such additional properties may include but are not limited to polydispersity, the shape of the particle size distribution, and attractive or repulsive interaction between the individual particles. These factors may also be tunable as part of the spray drying process.

Said another way, Table 1 also compares the tapped density of the sample 1 powder and of the sample 2 powder. Surprisingly, the tapped density of sample 1 is higher than that of sample 2 given that the analysis of particle density indicates sample 1 includes less dense particles than sample 2. In this way, tapped density of a powder is shown to not be equal to or proportional to a density of particles comprising the powder. The results shown in Table 1 unexpectedly show that individual particle densities may not be proportional to tapped density. As discussed above (e.g., with respect to FIG. 2), the tapped density property of the powder may be proportional with a glide force of a suspension prepared with the powder. For this reason, the tapped density of a powder may be used as material parameter when formulating an injectable suspension without demanding image analysis to determine particle solids and void percentages. Tapped density may incorporate additional properties such as, but not limited to polydispersity, the shape of the particle size distribution, and attractive or repulsive interaction between individual particles. As discussed herein, the factors affecting tapped density may be tunable by adjusting the spray drying process. For example, by adjusting one or more of relative saturation, Tₒᵤₜ, P_{dryer}, and excipient concentrations in the liquid feed.

As one example, tapped density of a dried powder collected from a spray drying system may be increased by increasing a concentration of solutes in a liquid feed (e.g., liquid feed 104) fed into a spray drier. As a further example, tapped density of dried powder collected from a spray dry system may be increased by drying at lower outlet temperatures, such as may be achieved by using a vacuum spray dryer (such as vacuum spray drier 100 of FIG. 1). Spray drying under vacuum has an additional benefit of exposing the API to lower temperatures than conventional spray drying, making spray drying possible for APIs otherwise considered to thermally unstable for spray drying.

Turning now to FIG. 6, a graph 600 showing glide force as a function of tapped density, similar to FIG. 2 is shown. Looking specifically at a first data point 602 it corresponds to a first spray solution dried using a conventional spray drier. A second data point 604 corresponds to the first solution dried using a vacuum spray drier. By drying under vacuum, the tapped density of the powders obtained from the same starting solution is increased. Turning now to a third data point 606 corresponding to a second solution dried using a conventional spray drier. The first solution is a low wt % solution while the second solution is a high wt % solution. By increasing a wt % of the solution, the tapped density of the dried particles from the solution is also increased. However, a solubility and stability of the API may limit a wt % of the solution. For example, the high wt % may be a maximum concentration of the API. A fourth data point 608 corresponds to the second solution dried using a vacuum spray drier. By using vacuum spray drying, a dried powder having a tapped density higher than what is achievable by increasing wt % of the spray dried solution alone may be produced. Additionally, an increase in tapped density from conventional compared to vacuum spray drying is greater when the tapped density is lower for the conventional spray drying. In this way, vacuum spray drying may be particularly relevant to solutions of APIs with low solubility or limits imposed on spray solution concentration due to API stability or for solution viscosity having a lower maximum tapped density when spray dried by conventional spray drying.

As described above, proteins may be spray dried from liquid feeds loaded with increasing amounts of protein to adjust an observed tapped density of the resulting dried particles. Tests shown below in Table 2 are from spray drying a test solution of bovine serum albumin (BSA) and sucrose in aqueous solution. BSA and sucrose are included at a 1:1 ratio by weight. A hydrophilic surfactant such as polysorbate 80 may also be included in the liquid feed.

TABLE 2
Spray drying tests of proteins

| | Test #1 | Test #2 | Test #3 | Test #4 | Test #5 | Test #6 |
|---|---|---|---|---|---|---|
| Solid wt. % | Low | High | High | High | High | High |
| L/G ratio | 0.0102 | 0.0102 | 0.0102 | 0.0102 | 0.0102 | 0.0147 |
| Atom. Press. | 1.0 | 1.0 | 1.0 | 1.0 | 2.0 | 2.0 |
| (barg) | | | | | | |
| In. Temp. | 44.5-60.2 | 48.6-52.8 | 56.9-59.8 | 19.4-19.6 | 19.3-19.6 | 22.4-30.2 |
| ° C. | | | | | | |
| Out. Temp. | 20.1-22.7 | 21.7-22.3 | 20.0-22.4 | 15.9-16.9 | 15.0-16.0 | 14.7-15.0 |
| ° C. | | | | | | |
| P_{dryer} (bar) | 0.383 | 0.375 | 0.308 | 0.297 | 0.292 | 0.297 |
| Predicted | 23.2 | 17.6 | 16.7 | 21.8 | 23.1 | 33.8 |
| RS % | | | | | | |
| Yield % | 80.5 | 76.9 | 79.6 | 52.5 | 68.9 | 47.9 |
| Tapped | 0.60 | 0.73 | 0.73 | 0.71 | 0.72 | 0.73 |
| density | | | | | | |
| (g/mL) | | | | | | |

Tests #1-#6 of Table 2 were all spray dried at P_{dryer} below atmospheric pressure over both low and high solids weight percent, both of which resulted in an acceptable percent yield and tapped density for an injectable formulation. Differences in dryer pressure between tests #1-#5 may also have relatively little impact on yield and tapped density. Comparing tests #3 and #4, a decrease in outlet temperature from about 21° C. to about 16° C. does result in a decreased yield, although tapped density is relatively constant. However, even the decreased yield of test #4 is greater than 50%. Comparing tests #4 and #5, increasing atomization pressure to decrease a droplet size also increases a percent yield without impacting the desired tapped density. Further, comparing tests #5 and #6, increasing L/G ratio also negatively impacts yield.

As a further example, additional tests of spray drying a 1:1 BSA: sucrose solution including buffer and polysorbate at the low solids wt. % were conducted and results are shown in Table 3 below. Such tests may be relevant models for proteins for which highly concentrated spray drying liquid feeds may not be possible. Higher concentrations may not be possible due to solubility and/or stability of the protein, or a viscosity of the liquid feed.

TABLE 3
Spray drying of protein at low concentrations

| | Test #1 | Test #2 | Test #3 | Test #4 | Test #5 |
|---|---|---|---|---|---|
| Solid wt. % | Low | Low | Low | Low | Low |
| L/G ratio | 2.02 | 2.02 | 2.02 | 2.02 | 2.92 |
| Atom. Press. | 1.0 | 1.0 | 1.0 | 2.0 | 2.0 |
| (barg) | | | | | |
| In. Temp. | 44.5-60.2 | 20.4-21.6 | 14.2-16.8 | 13.1-14.7 | 10.7-12.7 |
| ° C. | | | | | |
| Out. Temp. | 20.1-22.7 | 20.4-21.6 | 14.2-16.8 | 13.1-14.7 | 10.7-12.7 |
| ° C. | | | | | |
| P_{dryer} (bar) | 0.383 | 0.305 | 0.298 | 0.310 | 0.312 |
| Predicted | 23.2 | 18.5 | 27.0 | 28.1 | 49.1 |
| RS % | | | | | |
| Yield % | 80.5 | 81.8 | 70.7 | 71.1 | 67.7 |
| Tapped | 0.6 | 0.589 | 0.675 | 0.627 | 0.655 |
| density | | | | | |
| (g/mL) | | | | | |

Results of tests #1-test #6 shown in Table 3 confirm that spray drying under reduced pressure may result in powders having an acceptable tapped density (e.g., >0.45 g/mL) for an injectable formulation, even when a solid wt. % of the protein in the liquid feed is low. Comparing tests #1 to test #2 of Table 3, pressure decreases by about 78 mbar while yields and tapped density of the products stay substantially the same. Further, comparing test #2 to test #3 of Table 3, outlet temperature decreased resulting in an increase in tapped density of the product. Adjusting temperatures to lower temperatures without decreasing yield to below an acceptable amount may also produce high tapped density powders from low concentration liquid feeds. Additionally, comparing test #3 to test #4 of Table 3, atomization pressure increases resulting in smaller droplets. In this case, the smaller droplets may result in lower tapped density, although the tapped density may still be acceptable to include in an injectable formulation. Finally, comparing test #4 to test #5 of Table 2, increasing L/G by increasing a liquid feed rate may help to increase a throughput without substantially affecting yield or tapped density.

As a further example, lysozyme may be used as a model protein for a protein to be included in an injectable formulation. The tests described below in Table 4 were conducted by vacuum spray drying a liquid feed including lysozyme and sucrose in a 75:25 weight ratio. The liquid feed may also include a hydrophilic surfactant such as polysorbate 80.

TABLE 4
Spray drying of lysozyme under vacuum.

| | Test #1 | Test #2 | Test #3 | Test #4 |
|---|---|---|---|---|
| Solids wt. % | Low | Low | High | High |
| Inlet Temp. ° C. | 46.5-56.3 | 15.9-17.0 | 49.7-52.5 | 16.0-17.0 |
| Outlet Temp. ° C. | 20.1-21.5 | 10.8-13.8 | 19.8-21.5 | 11.7-12.2 |
| L/G ratio | 0.0102 | 0.0137 | 0.0102 | 0.0137 |
| Atom. Pressure | 1.0 | 1.0 | 1.0 | 2.0 |
| (barag) | | | | |
| P_{dryer} (bar) | 0.328-.399 | 0.309 | 0.319 | 0.313 |
| Predicted RS % | 20.4-24.8 | 50.9 | 16.5 | 39.9-28.0 |
| Yield (%) | 40.6 | 48.6 | 57.9 | 45.7 |
| Tapped density | 0.536 | 0.549 | 0.605 | 0.671 |
| (g/mL) | | | | |

Comparing test #1 and test #2 and test #4 and test #5 of Table 4 shows that decreasing outlet temperature also increases tapped density for a lysozyme product, similar to the BSA product. The effect occurs both when solids wt. % is high (test #5) and when solids wt. % is low (test #2). However, a larger increase in tapped density occurs with decreasing outlet temperature when solid wt. % is high than when the solid wt. % is low. Also similar to the BSA product, increasing the solid wt. % in the liquid feed also increases tapped density of the dried product as shown by comparing test #2 and test #3 of Table 4.

The technical effect of methods and systems disclosed herein is to produce spray dried powders having high tapped density. The spray dried particles having increased tapped density are used in preparing injectable formulations which may be delivered via a needle and syringe, sometimes with an auto-injector using a glide force of less than or equal to 100N or less than or equal to 50N. Increasing density of the spray dried powder included in an injectable formulation may result in a formulation that can be injected using a desired glide force without having to decrease a concentration of particles in the suspension or by adjusting the suspension or injection device in other ways which may not be cost effective.

The disclosure also provides support for an injectable suspension, comprising: a vehicle, and a spray dried powder having tapped density greater than 0.45 g/mL suspended in the vehicle at greater than or equal to 40 wt. %, and wherein the injectable suspension is injectable through a 27 gauge, ½ inch long 1 mL needle at an injection speed of approximately 1 mL/10 seconds using a glide force of less than or equal to 100N. In a first example of the system, the glide force is less than or equal to 50N. In a second example of the system, optionally including the first example, the vehicle is one or more of glyceryl tricaprylate/tricaprate, triacetin, propylene glycol dicaprylate/dicaprate, or ethyl oleate. In a third example of the system, optionally including one or both of the first and second examples, the spray dried powder includes a peptide or protein and wherein the spray dried powder includes an active pharmaceutical ingredient at a concentration greater than 50 wt %. In a fourth example of the system, optionally including one or more or each of the first through third examples, an average diameter of the spray dried powder is between 1-50 μm with a Dv90 of less than 100 μm. In a fifth example of the system, optionally including one or more or each of the first through fourth examples, a viscosity of the injectable suspension is less than or equal to 500 cP at a shear rate of 2000 s−1. In a sixth example of the system, optionally including one or more or each of the first through fifth examples, the spray dried powder is dried using a dryer pressure below 0.8 bar.

The disclosure also provides support for a method to produce a spray dried powder with high tapped density, comprising: selecting a maximum spray solution concentration for a liquid feed as defined by protein stability and solution viscosity, selecting a relative saturation and outlet temperature for spray drying, selecting a dryer pressure for spray drying the liquid feed, spray drying the liquid feed at the selected relative saturation, outlet temperature, and dryer pressure to obtain spray dried particles, and determining a tapped density of the spray dried powder, and in response to the tapped density of the spray dried powder below a threshold tapped density or a suspension of the spray dried powder dispensed above a threshold glide force, adjusting one or more of the maximum spray solution concentration, relative saturation, outlet temperature or dryer pressure. In a first example of the method, the method further comprises: preparing an injectable formulation with the spray dried powder, wherein the injectable formulation is deliverable through a 27 g, ½″ long needle with less than or equal to 100N dispensing force. In a second example of the method, optionally including the first example, the threshold tapped density is greater than 0.45 g/mL. In a third example of the method, optionally including one or both of the first and second examples, the method further comprises: determining a yield of the spray dried powder, and in response to the yield of spray dried powder below a threshold yield, adjusting one or more of the relative saturation, outlet temperature or dryer pressure. In a fourth example of the method, optionally including one or more or each of the first through third examples, the outlet temperature is less than 40° C. or less than 30° C. In a fifth example of the method, optionally including one or more or each of the first through fourth examples, the relative saturation is in a range of 5% to 20%. In a sixth example of the method, optionally including one or more or each of the first through fifth examples, the dryer pressure is less than 0.8 bar. In a seventh example of the method, optionally including one or more or each of the first through sixth examples, outlet temperature is less than 50° C., relative saturation is greater than 5% and dryer pressure is less than 0.8 bar. In a eighth example of the method, optionally including one or more or each of the first through seventh examples, the outlet temperature is less than 40° C. and the relative saturation is greater than 10%. In a ninth example of the method, optionally including one or more or each of the first through eighth examples, the outlet temperature is less than 35° C. and the dryer pressure is less than 0.8 bar. In a tenth example of the method, optionally including one or more or each of the first through ninth examples, the relative saturation is in a range of from 5% to 50%.

The disclosure also provides support for an injectable suspension, comprising: a vehicle, and a spray dried powder having tapped density greater than 0.45 g/mL suspended in the vehicle, and wherein a viscosity of the injectable suspension is less than or equal to 500 cP as a sheer rate of 2000 s−1. In a first example of the system, the viscosity of the injectable suspension is less than or equal to 250 cP. The disclosure also provides support for an injectable suspension, comprising: a vehicle, and a spray dried powder, wherein the spray dried powder is dried at a dryer pressure of less than 0.8 bar.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. An injectable suspension, comprising:
a vehicle; and
a spray dried powder having tapped density greater than 0.45 g/mL suspended in the vehicle at greater than or equal to 40 wt. %, and wherein the injectable suspension is injectable through a 27 gauge, ½ inch long 1 mL needle at an injection speed of approximately 1 mL/10 seconds using a glide force of less than or equal to 100N.

2. The injectable suspension of claim 1, wherein the glide force is less than or equal to 50N.

3. The injectable suspension of claim 1, wherein the vehicle is one or more of glyceryl tricaprylate/tricaprate, triacetin, propylene glycol dicaprylate/dicaprate, or ethyl oleate.

4. The injectable suspension of claim 1, wherein the spray dried powder includes a peptide or protein and wherein the spray dried powder includes an active pharmaceutical ingredient at a concentration greater than 50 wt %.

5. The injectable suspension of claim 1, wherein an average diameter of the spray dried powder is between 1-50 μm with a Dv90 of less than 100 μm.

6. The injectable suspension of claim 1, wherein a viscosity of the injectable suspension is less than or equal to 500 cP at a shear rate of 2000 s⁻¹.

7. The injectable suspension of claim 1, wherein the spray dried powder is dried using a dryer pressure below 0.8 bar.

8. A method to produce a spray dried powder with high tapped density, comprising:
selecting a maximum spray solution concentration for a liquid feed as defined by protein stability and solution viscosity;
selecting a relative saturation and outlet temperature for spray drying;
selecting a dryer pressure for spray drying the liquid feed;
spray drying the liquid feed at the selected relative saturation, outlet temperature, and dryer pressure to obtain spray dried particles; and
determining a tapped density of the spray dried powder, and in response to the tapped density of the spray dried powder below a threshold tapped density or a suspension of the spray dried powder dispensed above a threshold glide force, adjusting one or more of the maximum spray solution concentration, relative saturation, outlet temperature or dryer pressure.

9. The method of claim 8, further comprising, preparing an injectable formulation with the spray dried powder, wherein the injectable formulation is deliverable through a 27 g, ½″ long needle with less than or equal to 100N dispensing force.

10. The method of claim 8, wherein the threshold tapped density is greater than 0.45 g/mL.

11. The method of claim 8, further comprising determining a yield of the spray dried powder, and in response to the yield of spray dried powder below a threshold yield, adjusting one or more of the relative saturation, outlet temperature or dryer pressure.

12. The method of claim 8, wherein the outlet temperature is less than 40° C. or less than 30° C.

13. The method of claim 8, wherein the relative saturation is in a range of 5% to 20%.

14. The method of claim 8, wherein the dryer pressure is less than 0.8 bar.

15. The method of claim 8, wherein outlet temperature is less than 50° C., relative saturation is greater than 5% and dryer pressure is less than 0.8 bar.

16. The method of claim 8, wherein the outlet temperature is less than 40° C. and the relative saturation is greater than 10%.

17. The method of claim 8, wherein the outlet temperature is less than 35° C. and the dryer pressure is less than 0.8 bar.

18. The method of claim 8, wherein the relative saturation is in a range of from 5% to 50%.

19. An injectable suspension, comprising:
a vehicle; and
a spray dried powder having tapped density greater than 0.45 g/mL suspended in the vehicle, and wherein a viscosity of the injectable suspension is less than or equal to 500 cP as a sheer rate of 2000 s⁻¹.

20. The injectable suspension of claim 19, wherein the viscosity of the injectable suspension is less than or equal to 250 cP.
